(19) United States
(12) Patent Application Publication
(10) Pub. No.: US 2007/0112343 A1
(43) Pub. Date: May 17, 2007

(54) Surgical tip device with retractable sheath and methods for using same

(76) Inventors: Hans Mische, Saint Cloud, MN (US); Stephen M. Zappala, Andover, MA (US)

Correspondence Address: Mirick, O'Connell, DeMallie & Lougee, LLP, 100 Front Street, Worcester, MA, 01608, US

(21) Appl. No.: 11/337,224

(22) Filed: Jan. 20, 2006

(51) Int. Cl. A61B 18/14

(52) U.S. Cl. 606/41; 606/16; 606/20

(57) ABSTRACT

A surgical tip device, that generally includes a proximal handle; a distal surgical member, having a surgical tip, and at least temporarily fixed to the handle; and a retractable sheath that at least partially covers the surgical tip when the sheath is not retracted and the surgical tip is not in use.

### FIELD OF THE INVENTION

The invention relates to devices, and methods for using the devices, for electrosurgery and more specifically to a surgical tip device that has a retractable sheath that reduces the risk of injury to patients and medical personnel.

### BACKGROUND OF THE INVENTION

Radiofrequency (RF) and laser sources are commonly employed in surgery through a hand held activator, that are capable of delivering a variety of currents and energy levels for coagulation, cutting and tissue stimulation. The terminal section of the hand held delivery system applies the energy typically through a detachable tip. Such tips are available in many shapes and sizes. The tips used for conventional surgery are either blunt or needlepoint. The needlepoint electrode offers significant advantages over larger and/or blunt tipped electrodes. The fine point of a needle improves accuracy to dessicate, diminishes RF scattering during coagulation and increases the versatility of electrosurgery, especially within the pediatric surgical population. Moreover, the widespread acceptance of minimally invasive surgical techniques and laparoscopy will usher in wider applications for needle tip delivery systems.

However, needlepoint electrodes have several drawbacks. Significant morbidity to both the patient and the entire surgical team is associated with the needlepoint electrode. The needlepoint is sharp enough to penetrate a vital organ or major vascular structure if inadvertently dropped into an open incision. The surgeon and allied health personnel are also at significant risk for puncture wounds during surgical procedures. As such, in view of the further risk of contracting HIV and Hepatitis viruses through puncture wounds, many operating rooms avoid using the needlepoint system.

In addition to the health risks, surgical tips, such as the fiber optic tips associated with surgical laser devices, are somewhat fragile and susceptible to damage if handled roughly or scratched on their surgically active surfaces.

Some devices are known that include either retractable electrodes, such as the jaw-like retractable blades disclosed in U.S. Pat. No. 5,456,684 and the complex retractable, adjustably angled needlepoint devices disclosed in U.S. Pat. Nos. 5,366,490 and 5,456,684. However, these devices are limited to very specific uses, they are complex and are not adapted for delivering finely directed and easily manipulated energies currently available for surgical uses.

### SUMMARY OF THE INVENTION

It is therefore a primary object of this invention to provide a surgical tip that is accurate, versatile and safe, and to provide methods for using the tip.

It is a further object of this invention to provide a safe, surgical tip that is particularly suited for use within the pediatric surgical population.

It is a further object of this invention to provide a surgical tip that is adapted to deliver coagulating and cutting currents.

It is a further object of this invention to provide a retractable sheath for surgical tips that is disposable and enhances the safety of electro or laser surgery to both personnel and patients.

The retractable sheath of the surgical tip of the invention was designed to provide a beneficial alternative to the deployable needlepoint electrode disclosed in U.S. Pat. No. 6,569,161B2. The deployable needlepoint electrode disclosed in the '161 patent offers many of the same advantages as the subject invention, however, the applications will likely differ somewhat depending on the surgical conditions. In the subject invention,.the sheath protecting the surgical tip is retracted back from the tip towards the handle of the device when in use as opposed to the deployable needle disclosed in the '161 patent that is pushed out from a fixed sheath to expose the electrode tip when in use. In the subject invention the needle remains fixed in position relative to the handle and the sheath is moved back and forth. The subject invention also includes the combination if desired in which the sheath and the surgical tip may both be adjusted or otherwise movable.

A preferred embodiment of the surgical tip device of the invention generally comprises: a proximal handle; a distal surgical member, having a surgical tip, and at least temporarily fixed to the handle; a retractable sheath that at least partially covers the surgical tip; and a means for retracting the sheath backwards from the tip towards the handle to at least partially uncover the tip. The device may further comprise a means located on the handle for activating the surgical tip. The surgical tip may comprise one or more tips such as, but not limited to, a needle point electrode, a blunt electrode, a fiber optic laser probe, a cryogenic probe, a paddle, a ball, and a loop The means for retracting may take a variety of forms including but not limited to a plurality of threads on the sheath and a corresponding plurality of threads on the handle; and/or one or more tension spring members between the sheath and the handle.

The sheath may also comprise a contact-activating member and the handle may likewise comprise one or more corresponding activation contacts. The device preferably further comprises a safety means for preventing unintentional activation of the surgical tip, wherein the safety means may comprise a housing on the handle that at least partially encloses the means for activating the surgical tip.

Another preferred embodiment of the surgical tip device of the invention generally comprises: a proximal handle having an electrode receptacle and a sheath receptacle having a plurality of threads; a distal electrode surgical member, having a distal electrode surgical tip, and a proximal electrode end that is at least temporarily fixed in the electrode receptacle; a retractable sheath at least partially covering the tip and having a plurality of threads corresponding to the threads of the sheath receptacle ; and a means for activating the surgical tip. The sheath receptacle threads may be provided on an inside surface of the handle, in which case the retractable sheath threads are preferably provided on an outside surface of the retractable sheath.

A preferred method of the invention for using a surgical tip device, generally comprises the steps of: providing a surgical tip comprising, a proximal handle; a distal surgical tip, having a surgically active tip, and at least temporarily fixed to the handle; a retractable sheath that at least partially covers the surgically active tip; and a means for retracting the sheath backwards from the tip towards the handle to at least partially uncover the tip; retracting the sheath to uncover the tip; and activating the surgically active tip.

The means for retracting may comprise a means for at least partially rotating the sheath around the surgical tip, wherein the means for rotating may comprise a plurality of threads provided on the sheath and a plurality of corresponding threads provided on the handle; and/or one or more grooves on the sheath and a detent on the handle.

The surgical tip device may further comprise a means for activating the surgically active tip wherein the means for activating then preferably comprises one or more contact activating members on the sheath and one or more activation contacts on the handle. The device used in the method may still further comprise a safety means for preventing unintended activation of the currents, wherein the safety means comprises one or more detents on the sheath and one or more activation members at least partially enclosed in a housing on the handle. The housing may comprise an opening through which the detent is adapted to engage the activation member when the sheath is retracted.

Another preferred method for using the surgical tip device of the invention that is adapted to deliver adjustable levels of energy to a surgical site, generally comprises the steps of, providing a surgical tip device comprising, a proximal handle; a distal surgical member, having a surgically active region, and at least temporarily fixed to the handle; a sheath that at least partially covers the surgically active region; and a means for adjusting the relative positions of the sheath and the surgically active region, backwards to and forwards from the handle, to variably expose the surgically active region of the distal surgical member; activating the surgical tip; and adjusting the level of energy at the surgical site by variably exposing one or more areas of the surgically active region of the distal surgical member.

The invention is adapted to deliver laser or electro energies at its tip that are protected within a retractable sheath that is retracted when the surgical tip device is in use. The invention is particularly suited for incorporation into a handle hand device much like a ballpoint pen except that the sheath is retracted rather than the tip deployed. A variety of means are disclosed for retracting the sheath.

### BRIEF DESCRIPTION OF THE DRAWINGS

Other objects, features and advantages will occur to those skilled in the art from the following description of the preferred embodiments and the accompanying drawings in which:

FIG. 1A is a side view of a preferred embodiment of the surgical tip device of the invention showing the sheath in a non-retracted position;

FIG. 1B is a side view of the embodiment shown in FIG. 1A showing the sheath in a retracted, surgical position;

FIG. 2A is a side view of another preferred embodiment of the surgical tip device of the invention showing the sheath in a non-retracted position;

FIG. 2B is a side view of the embodiment shown in FIG. 2A showing the sheath in a retracted, surgical position;

FIG. 3 is a side view of another preferred embodiment of the surgical tip device of the invention showing the sheath in a non-retracted position;

FIG. 4 is a side view of the embodiment shown in FIG. 3 showing the sheath in a retracted, surgical position;

FIG. 5 is side-by-side view of another embodiment of the sheath and the forward section of the handle showing another means for retracting the sheath of the invention;

FIG. 6 is side-by-side view of another embodiment of the sheath and the forward section of the handle showing another means for retracting the sheath of the invention;

FIG. 7 is a side view of another embodiment of the surgical tip and sheath of the invention with the sheath fully retracted and the sheath fully enclosing the surgical tip;

FIG. 8 is a side view of the embodiment of the surgical tip and sheath of the invention shown in FIG. 7 with the sheath fully retracted and the sheath partially retracted.

### DETAILED DESCRIPTION OF THE PREFERRED EMBODIMENTS AND METHODS

The invention generally features a sheath that protects a surgical tip device, such as a needlepoint electrode or laser tip, by surrounding the tip while the tip is not in use and then is retracted backwards from the tip towards the handle away from the tip to expose the tip when the surgical tip is in use. The preferred embodiments of the device of the invention are shown in the Figures.

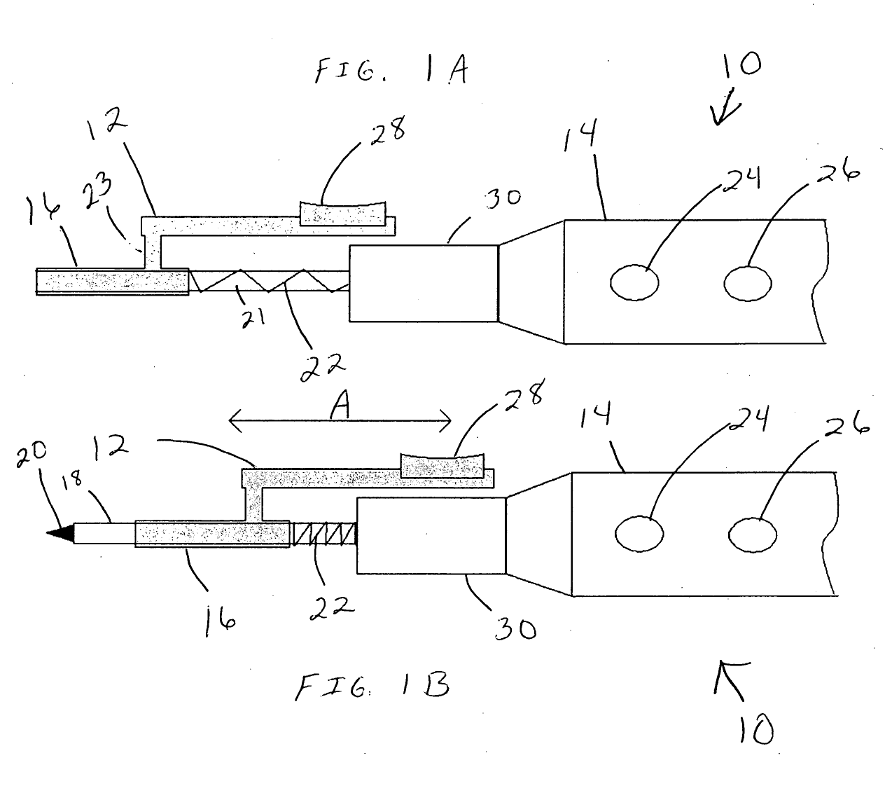

The first described embodiment is shown and generally referred to in FIGS. 1A and 1B as device 10. As with all of the embodiments described herein, device 10 can be readily inserted into currently available hand held systems, connected to a generator and then thrown away after a single use.

Device 10 comprises proximal handle 14 having an electrode receptacle 30 into which the proximal end of electrode 21 is inserted and at least temporarily fixed in place. Electrode 21 is one example of the surgical tip of the invention. Other surgical tips include but are not necessarily limited to, needles, paddles, ball-ends, lasers, cryogenic tips, and probes. The surgical tip device of the invention may be used in all surgical specialities such as, but not limited to, urological, endoscopic, laparoscopic, neurology, cardiology, orthopedic, gynecological, colorectal, vascular, and opthalmic. The device of the invention may also include a light source and/or an irrigation canal proximate the surgical tip end.

Device 10 further comprises needle sheath 16 having a finger grip 12 with a finger tab 28. Finger tab 28 may be an ergonomically molded depression in grip 12 as shown in FIG. 1A or other suitable gripping means such as one or more raised asperities on the surface of grip 12. Device 10 is shown with the components concentrically or telescopically arranged so that the outside diameter of the needle 18 with needle or electrode tip 20 is smaller than the inside diameter of sheath 16. In turn, sheath 16 has an outside diameter that is smaller than the inside diameter of receptacle 30. The outside surface of receptacle 30 then increases gradually or frustoconically to a larger diameter and more ergonomically sized handle 14 for gripping. Device 10 further comprises bridge 23 that extends out laterally from sheath 16 to grip 12 to enable grip 12 to be pull backwards towards handle 14. Depending on the distance that grip 12 is pulled backwards, bridge 23 allows grip 12 to clear the outside surface of handle 14.

Device 10 also comprises tension spring 22 that is seated concentrically around needle electrode 21 between the rearward end of sheath 16 and the forward end of receptacle 30. Tension spring 22 places forward pressure on sheath 16 to maintain sheath 16 in a non-retracted position when not actively being pulled backwards. Inner shoulders (not shown) may be provided on the inner forward end of sheath 16 and the inner rearward end of receptacle 30 against which spring 22 may be seated inside the inner tubular sides of sheath 16 and receptacle 30. Sheath 16 may further comprise a second telescoping member (not shown) between the forward section of sheath 16 and handle 14 to fully enclose needle electrode 21 when spring 22 is in a relaxed state and needle tip 20 is fully enclosed as shown in FIG. 1A. When grip 12 is pulled backwards in the direction of arrow A towards handle 14, sheath 16 retracts from the distal end of needle 18 to expose needle tip 20 as shown in FIG. 1B.

Handle 14 is also provided with two buttons 24 and 26 for engaging, respectively, the cutting and coagulating currents carried over electrode 21 to the surgical site. Buttons 24 and 26 may be provided with a safety to prevent them from engaging the electrical current unless sheath 16 is in a retracted position.

The retracting sheath not only protects the surgical tip from damage and users from injuries, as importantly, it also enables the user to control the level and intensity of the energy delivered at the surgical site. For example, the level and density of the electrical energy delivered to the surgical site using a surgical electrode may be increased or decreased, respectively, by decreasing or increasing the exposed area of the surgically active tip. The intensity or power at the surgical tip may be increased by reducing the active area at the distal end of the surgical tip through which the same amount of electrical energy is delivered at the surgical site. The same is true of laser energy delivered through an optical fiber tip.

The adjustment of power may also be achieved by moving the surgical tip relative to the sheath, as opposed to moving the sheath relative to the surgical tip. A number of means for retracting or moving the needle relative to the sheath are described in U.S. Pat. No. 6,569,161B2 issued on May 27, 2003 to S. Zappala and is hereby incorporated by reference to described such means.

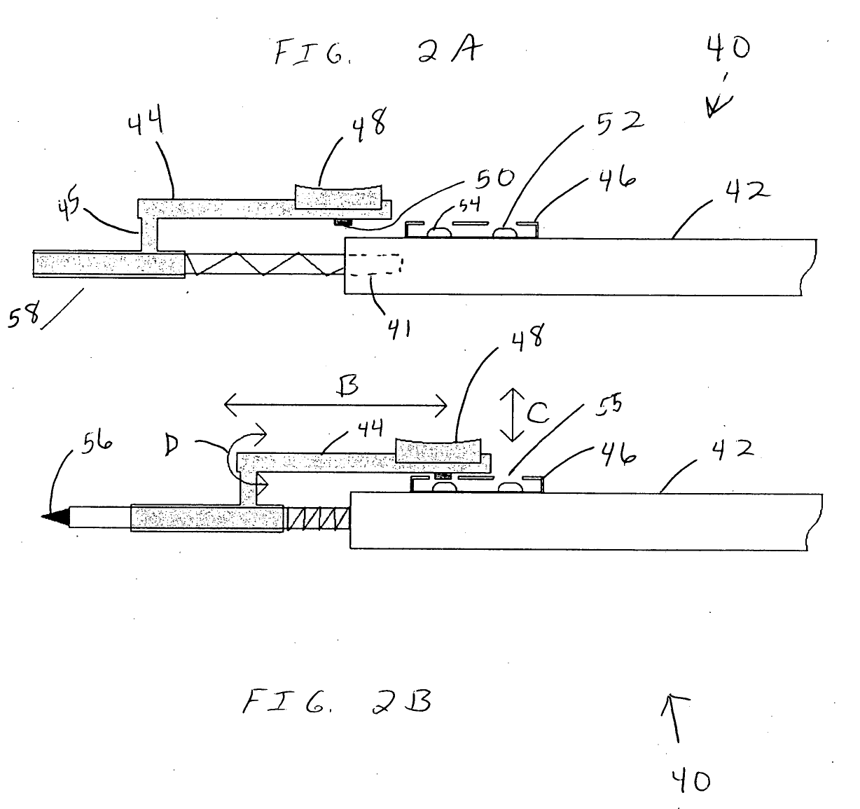

Another embodiment of the needlepoint electrode device of the invention is shown in FIGS. 2A and 2B and is generally referred to as device 40. Similar to device 10, needlepoint electrode device 40 comprises proximal handle 42 having an inner electrode receptacle. 41 into which the proximal end of electrode is inserted and at least temporarily fixed in place. Device 40 further comprises needle sheath 56 having a finger grip 44 with a finger tab 48 integrally and ergonomically molded into grip 12 as shown in FIG. 2A. Device 40 is shown with the components concentrically or telescopically arranged so that the outside diameter of the needle 18 with needle or electrode tip 20 is smaller than the inside diameter of sheath 16. In turn, sheath 16 has an outside diameter that is smaller than the inside diameter of receptacle 30. Device 40 further comprises bridge 45 that extends out laterally from sheath 58 to grip 44 to enable grip 44 to be pull backwards towards handle 42 in the direction of arrow B and turned in the direction of arrow D.

Bridge 45, in this embodiment allows grip 44 to clear the outside surface of button housing 46. Button housing 46 acts as a type of safety to prevent buttons 52 and 54 from being activated when sheath 58 is not retracted. Grip 44 further comprises detent 50, which is adapted to be inserted into opening 55 in the top of button housing 46 to engage buttons 52 and 54 when sheath 58 is retracted. Depending on the height of bridge 45 or the thickness of detent 50, bridge 45 may be elastomeric to enable grip 44 to be depressed in the direction of arrow C so that detent 50 engages buttons 52 and 54 when inserted into openings 55. The elasticity of bridge 45 will depend on the material used but should offer a degree of resistance when depressed to ensure that detent 50 spontaneously disengages from buttons 52 and 54 when finger tab 48 is released. If continuous current is desired without having to continuously depress tab 48, a releasable or resistive catch may be provided on detent 50 or housing 46, or any other suitable surface, to temporarily maintain detent 50 in position against one of the buttons.

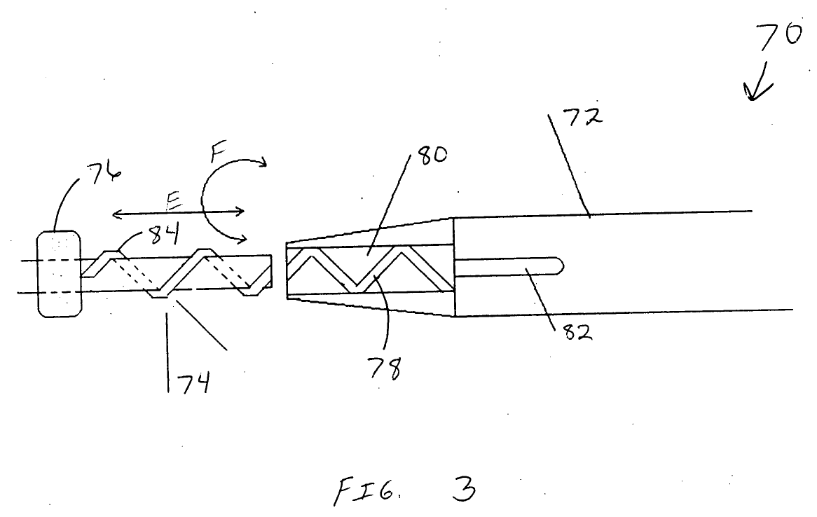

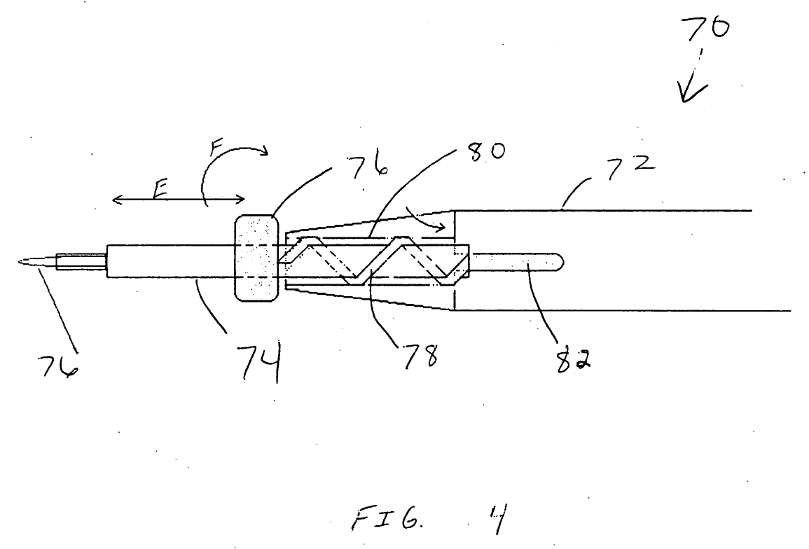

Another preferred embodiment is shown in FIGS. 3 and 4 and generally referred to as needlepoint electrode device 70. Device 70 comprises a pen body or handle 72 with a threaded electrode receptacle 80 and a correspondingly threaded sheath 74. Handle 72 is also provided with a non-threaded receptacle 82 into which the proximal end of the electrode is inserted and at least temporarily fixed or otherwise held is place. Sheath 74 is provided with a finger knob 76 that is used to provide a grip on sheath 74 to rotate sheath 74. Sheath 74 is rotated is the direction of arrow F to move sheath 74 forward and backwards in the direction of arrow E. Threads 84 are provided on the outside surface-of sheath 74 that correspond to threads 78 provided on the inside surface of threaded receptacle 70. When sheath 74 is rotated in one direction, sheath 74 retracts backwards from the distal end of the needle tip towards the handle to expose the surgically active needle electrode tip during surgical procedures. When sheath 74 is rotated in the opposite direction, sheath 74 moves forward again to recover tip 76 of the needle electrode. The extent to which sheath 74 is rotated in either direction of arrow E will determine the density of the energy delivered at the surgical tip assuming that the current level remains the same. The amount and density of the energy delivered may be adjusted by varying the area of the exposed surgical tip and by raising and lowering the current at the generator or other energy source. Such adjustments, either at the surgical tip or at the generator, can be used to alternate between coagulating and cutting modes.

Although buttons 24, 26, 52, and 54 are described as a means for activating the RF current of the device and may be used with any of the embodiments described, other suitable means for activating may be used. Such means include but are not limited to one or more switches located on the device itself or at location remote from the device, or contacts provided on opposing inner or outer surfaces of the retractable sheath and the handle. In the latter instance, activation is achieved when the sheath is retracted and a contact on the sheath touches one or more corresponding contacts on the handle or handle receptacle.

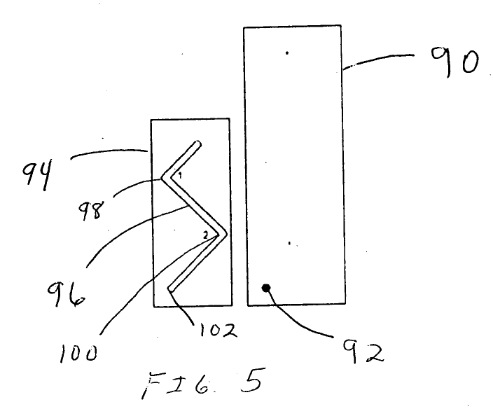

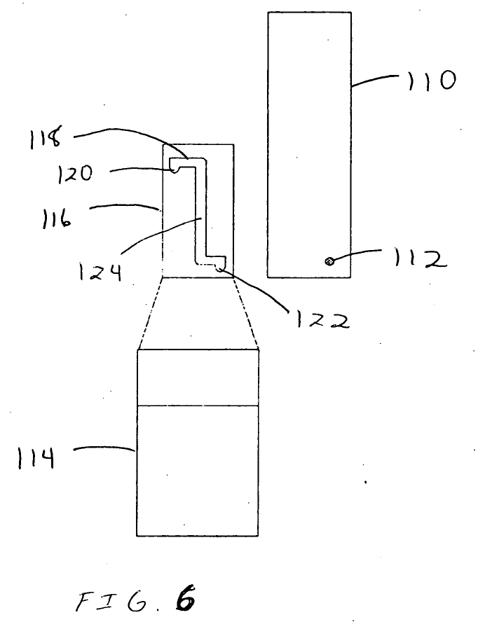

Although not meant to be limiting, FIGS. 5 and 6 illustrate two other means for retracting the sheath of the invention. Sheath 90, shown in FIG. 5, comprises a tab or detent 92, and handle receptacle 94 comprises a corresponding notched groove 96 on the inner surface. If desired, the detent and groove may be provided instead on the inside surface of the receptacle and the outside surface of the sheath, respectively. To retract sheath 90, sheath 90 is rotated backward along groove 96. In the embodiment shown, sheath 90 may be adjusted to three different positions at stops 98, 100 and 102. Any number of position stops may be provided as needed.

Similarly, sheath 110, shown in FIG. 6, comprises a detent 112, and handle receptacle 114 comprises a corresponding notched groove 124. In the non-retracted position, tab or detent 112 is seated in stop 120. To retract sheath 110, the sheath is pulled slightly forward, to release the detent from 120, rotated counterclockwise along lateral groove 118, then pulled backwards toward the handle until it butts up against the proximal end of groove 124, and then rotated counterclockwise again and released so that detent is seated in stop 122. To move sheath 110 forward again to protect the needle electrode tip the reverse steps are taken. Sheath 110 is pushed forward slightly to release the detent from stop 122, then rotated clockwise, the pushed forward along groove 124 until it butts up against groove 118, and rotated again clockwise and released to seat again in stop 120.

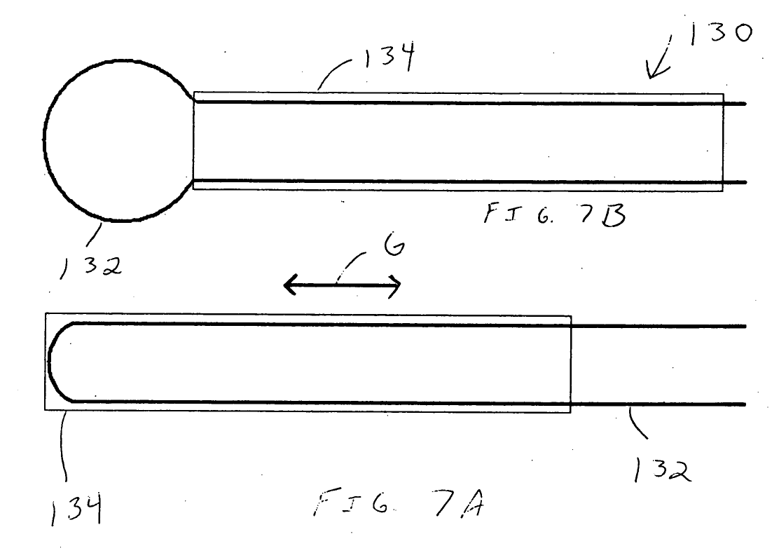

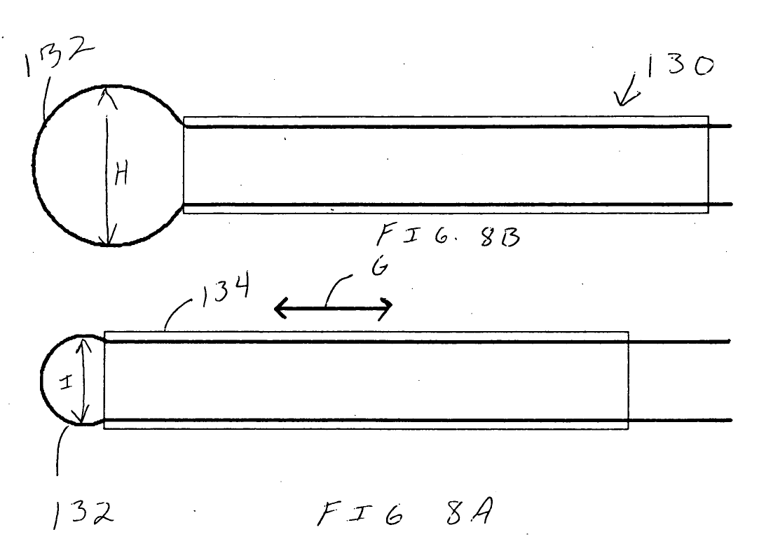

Another embodiment of the surgical tip and sheath of the invention are shown in FIGS. 7 and 8. The distal end of device 130 comprises metal surgical loop 132 and retractable sheath 134. In FIG. 7B, the surgical tip of loop 132 is shown fully exposed when sheath 134 is fully retracted in the direction of arrow G. In FIG. 7A, the surgical tip of loop 132 is fully enclosed and protected when sheath 134 is in a closed position. As shown in FIGS. 8A and 8B, the diameter, and therefore size, of the distal end of surgical loop 132 may be adjusted by only partially retracting sheath 134. As shown in FIG. 8B, the diameter H of loop 132 is at its maximum when sheath 134 is fully retracted. Yet, as shown in FIG. 8A, the diameter I of loop 132 is reduced when sheath 134 is only partially retracted. By adjusting the extent to which sheath 134 is retracted, either or both the density of energy delivered at the surgical site and the size of the loop may be modified to suit the surgical application.

In addition to the various means for retracting the sheath that are shown in the drawings other means, although not limiting, may comprise corresponding elastomeric materials on the outside of the sheath and the inside of the handle, and/or any other materials or surface asperities that would provide a frictional or mechanical means for retracting the sheath from the needle electrode tip and at least temporarily maintaining the sheath in a retracted position.

The needlepoint electrode of the invention has a surgically active tip that is adapted to deliver both coagulating and cutting currents. The device of the invention is preferably insulated over 80% of its electrode surface to reduce scatter and the inadvertent delivery of current through tissue that may come into contact with the shaft of the device. The sheath, in addition to any of the other components of the device of the invention, may be made from electrically and/or thermally insulating materials to reduce or prevent electrocution or burns.

The method of the invention for using a surgical radio frequency needlepoint electrode begins by providing a surgical radio frequency electrode device, such as devices 10, 40 and 70, and inserting the device into a hand held device that is connected to an RF generator. The surgically active region of the needlepoint electrode is then exposed by retracting the needlepoint sheath from the needlepoint tip by pulling backwards on the sheath, as in devices 10 and 40 or by turning the sheath to rotate and move the sheath backwards as in device 70. After the electrode top is exposed, the surgeon activates the surgically active region to the tip by initiating a current for cutting and/or coagulating through the electrode by depressing the appropriate button or otherwise engaging the activating contact. After the tissues are cut, ablated and/or coagulated as needed, the surgically active region of the needlepoint electrode covered by releasing the pressure on the grip of devices 40 and 70 or by rotating the sheath of device 70 forward to allow the sheath to surround and protect the distal tip of the needlepoint electrode.

Although specific features of the invention are shown in some drawings and described in connection with one embodiment, and not others, this is for convenience only as some feature may be combined with any or all of the other features in accordance with the invention.

Other embodiments will occur to those skilled in the art and are within the following claims:

What is claimed is:

1. A surgical tip device, comprising,
a proximal handle;
a distal surgical member, having a surgical tip, and at least temporarily fixed to said handle;
a retractable sheath that at least partially covers said surgical tip; and
a means for retracting said sheath backwards from said tip towards said handle to at least partially uncover said tip.

2. The device of claim 1, wherein said means for retracting comprises a plurality of threads on said sheath and a corresponding plurality of threads on said handle.

3. The device of claim 1, further comprising a means located on said handle for activating said surgical tip.

4. The device of claim 1, wherein said means for retracting comprises one or more tension spring members between said sheath and said handle.

5. The device of claim 1, wherein said sheath further comprises a contact activating member and said handle comprises one or more activation contacts.

6. The device of claim 1, further comprising a means located on said handle for activating said surgical tip and a safety means for preventing unintentional activation of said surgical tip.

7. The device of claim 6, wherein said safety means comprises a housing on said handle that at least partially encloses said means for activating said surgical tip.

8. The device of claim 1, wherein said surgical tip comprises one or more tips selected from a group consisting of a needle point electrode, a blunt electrode, a fiber optic laser probe, a cryogenic probe, a paddle, a ball, and a loop.

9. A surgical tip device, comprising,
a proximal handle having an electrode receptacle and a sheath receptacle having a plurality of threads;
a distal surgical member, having a distal surgical tip, and a proximal electrode end that is at least temporarily fixed in said electrode receptacle;
a retractable sheath at least partially covering said surgical tip and having a plurality of threads corresponding to said threads of said sheath receptacle;
and a means for activating said surgical tip.

10. The device of claim 9, wherein said sheath receptacle threads are provided on an inside surface of said handle and said retractable sheath threads are provided on an outside surface of said retractable sheath.

11. A method for using a surgical tip device, comprising the steps of,
providing a surgical tip device comprising,
a proximal handle;
a distal surgical member, having a surgical tip, and at least temporarily fixed to said handle;
a retractable sheath that at least partially covers said tip; and
a means for retracting said sheath backwards from said tip towards said handle to at least partially uncover said tip;
retracting said sheath to uncover said tip; and
activating said surgical tip.

12. The method of claim 11, wherein said means for retracting comprises a means for at least partially rotating said sheath around said surgical tip.

13. The method of claim 12, wherein said means for rotating comprises a plurality of threads provided on said sheath and a plurality of corresponding threads provided on said handle.

14. The method of claim 12, wherein said means for rotating comprises one or more grooves on said sheath and a detent on said handle.

15. The method of claim 11, further comprising a means for activating said surgical tip.

16. The method of claim 15, wherein said means for activating comprises one or more contact activating members on said sheath and one or more activation contacts on said handle.

17. The method of claim 11, wherein said means for retracting comprises one or more tension spring members between said sheath and said handle.

18. The method of claim 11, further comprising a means for activating said surgical tip and a safety means for preventing unintended activation of said surgical tip.

19. The method of claim 18, wherein said safety means comprises one or more detents on said sheath and one or more activation members at least partially enclosed in a housing on said handle.

20. The method of claim 19, wherein said housing comprises an opening through which said detent is adapted to engage said activation member when said sheath is retracted.

21. A method for using a surgical tip device that is adapted to deliver adjustable levels of energy to a surgical site, comprising the steps of,
providing a surgical tip device comprising,
a proximal handle;
a distal surgical member, having a surgically active region, and at least temporarily fixed to said handle;
a sheath that at least partially covers said surgically active region; and
a means for adjusting the relative positions of said sheath and said surgically active region, backwards to and forwards from said handle, to variably expose said surgically active region of said distal surgical member;
activating said surgical tip; and
adjusting said level of energy at said surgical site by variably exposing one or more areas of said surgically active region of said distal surgical member.

22. The method of claim 21, wherein said surgical tip comprises one or more tips selected from a group consisting of needle point electrode, a blunt electrode, a fiber optic laser probe, a cryogenic probe, a paddle, a ball, and a loop.
